(19) United States
(12) Patent Application Publication
(10) Pub. No.: US 2009/0082304 A1
(43) Pub. Date: Mar. 26, 2009

(54) Methods of Treating Hematological Malignancies with Nucleoside Analog Drugs

(75) Inventors: Kulsoom Ghias, Chicago, IL (US); Chunguang Ma, Champaign, IL (US); Varsha Gandhi, Houston, TX (US); Leonidas C. Platanias, Glencoe, IL (US); Nancy L. Krett, Elk Grove Village, IL (US); Steven T. Rosen, Evanston, IL (US)

Correspondence Address: OPPENHEIMER WOLFF & DONNELLY LLP, 45 SOUTH SEVENTH STREET, SUITE 3300, MINNEAPOLIS, MN, 55402, US

(73) Assignees: NORTHWESTERN UNIVERSITY, Evanston, IL (US); THE BOARD OF REGENTS OF THE UNIVERSITY OF TEXAS SYSTEM, AUSTIN, TX (US)

(21) Appl. No.: 11/719,121

(22) PCT Filed: Nov. 14, 2005
(86) PCT No.: PCT/US05/41037

§ 371 Date: May 11, 2007

Related U.S. Application Data
(60) Provisional application No. 60/626,862, filed on Nov. 12, 2004.

(51) Int. Cl. A61K 31/7076; A61K 31/70; G01N 33/53; C12Q 1/02; C12Q 1/48; C12Q 1/42; C12Q 1/37; A61P 35/00

(52) U.S. Cl. 514/46; 514/43; 436/86; 435/29; 435/15; 435/21; 435/24

(57) ABSTRACT

The present invention provides methods of treating hematological malignancies, including multi-drug resistant malignancies, with 8-amino-adenosine and variants thereof. Also encompassed by the present invention is a method of predicting the response of a patient diagnosed with a hematological malignancy to treatment with a nucleoside analog and a method of screening candidate drugs for efficacy in treating hematological malignancies.

### RELATED APPLICATION DATA

This application claims the benefit of U.S. Provisional Application 60/626,862, filed Nov. 12, 2004, which is herein incorporated by reference in its entirety.

### FIELD OF INVENTION

This application relates to methods for treating hematological malignancies with nucleoside analog drugs, such as 8-amino-adenosine.

### BACKGROUND OF INVENTION

Leukemia, lymphoma, and myeloma are hematological malignancies, also known as blood-related cancers, which collectively rank fifth among cancers in incidence and second among cancers in mortality in the United States. Despite improvements in treatments, significant challenges remain. For instance, current treatments frequently result in adverse events including secondary malignancies, organ dysfunction (cardiac, pulmonary and endocrine), long lasting neuropyschological and psychosocial issues, as well as problems associated with quality of life. Although treatment may lead to long-term remission and a cure for some, for many, hematological malignancies are chronic diseases that ultimately result in death. The five year survival rate, for example, for Hodgkin's disease is 83%, for Non-Hodgkin's lymphoma is 53%, for all leukemias is 45%, for multiple myeloma is 29%, and for acute myelogenous leukemia is 14% (George Dahlman on behalf of the Leukemia & Lymphoma Society, before U.S. Senate Committee on Appropriations, Defense Subcommittee, May 15, 2003). Thus, a significant need remains for new treatments for these diseases.

Myeloma, also referred to as multiple myeloma (MM), is a B cell lymphoproliferative disorder in which malignant plasma cells accumulate in the bone marrow. In a normal person, plasma cells account for less than 5% of the cells. However, in a patient suffering from multiple myeloma, plasma cells can comprise more than 10% of the cells present. Most forms of myeloma metastasize quickly to multiple sites in the bone marrow and surrounding bone. Myeloma plasma cells, referred to as myeloma cells, produce growth factors such as vascular endothelial growth factor (VEGF) which promotes angiogenesis. Myeloma cells also have special adhesion molecules on their surface allowing them to target bone marrow where they attach to stromal cells and produce cytokines such as interleukin 6 (L-6), receptor for activation of NF-κB (RANK) ligand, and tumor necrosis factor (TNF). The cytokines stimulate the growth of myeloma cells and inhibit apoptosis, leading to proliferation of myeloma cells and ultimately bone destruction.

Myeloma cells within a person suffering from the disease are identical and produce the same immunoglobulin (IgG, IgA, IgD, or IgB), called monoclonal (M) protein or paraprotein, in large quantities. Although the specific M protein varies from patient to patient, it is almost always the same in any one patient. In two-thirds of all cases, the serum immunoglobulin belongs to the IgG class, the other one-third is usually IgA. In rare cases, IgE or IgD or a mixture of the two occur. Serum or urine electrophoresis can be used to identify M proteins. Another important diagnostic feature of MM is the presence of light chains, referred to as Bence-Jones proteins, in the urine. Bence-Jones proteins comprise free κ or λ light chains but never both (Haen, 1995, Principles of Hematology).

MM frequently results in bone destruction of the axial skeleton marked by pain and fracture. Amyloidosis associated with multiple myeloma is a relatively common finding. Renal failure, hypercalcemia, anemia, increased susceptibility to bacterial infection, and impaired production of normal immunoglobulin are also common clinical manifestations of the disease.

MM represents approximately 1% of all cancers and 2% of all cancer deaths. There is no cure for this blood cancer and median survival from diagnosis is 3 to 4 years with conventional therapy. Although high-dose chemotherapy and stem cell transplantation are successful in inducing remission, patients eventually relapse and/or develop drug-resistant disease (Jemal et al., 2004, CA Cancer J. Clin. 54: 8-29; Sirohi et al., 2004, Lancet. 363: 875-87).

Cytotoxic purine and pyrimidine nucleoside derivatives were among the earliest chemotherapeutic agents successfully introduced for anti-tumor therapy and belong to a pharmacologically diverse family containing cytotoxic, anti-viral and immunosuppressive agents. Although some nucleoside analogs are currently used for the treatment of acute and chronic hematological malignancies, these analogs have not exhibited sufficient activity in vitro or have failed in clinical trials to justify continued clinical evaluation for treatment of MM (Hjertner et al., 1996, Leukemia Research 20: 155-60; Oken, 1992, Cancer. 70: 946-8; Plunkett et al., 2001, Cancer Chemother. Biol. Response Modif. 19: 21-45; Nagourney et al., 1993, Br. J. Cancer. 67: 10-14).

There is a need for drugs that target molecules involved in the disease process. MAPKs are signaling molecules and are regulated through a three-tiered phosphorylation cascade. MAPKs are inactivated when dephosphorylated at threonine and/or tyrosine residues by cellular phosphatases (Ono, 2000, Cell Signal. 12: 1-13; Chang et al., 2001, Nature. 410: 37-40). Through the phosphorylation cascade, MAPKs coordinate diverse extracellular stimuli and regulate fundamental cellular processes including changes in gene expression, proliferation, differentiation, cell cycle arrest and apoptosis.

The Akt kinase pathway is another signaling cascade that plays a pivotal role in cell growth and survival. Akt substrates are involved in several cellular processes including regulation of protein synthesis, metabolism, homeostatic, cell cycle, cell survival and growth, and apoptosis (Franke et al., 2003, Oncogene. 22: 8983-98; Scheid et al., 2003, FEBS Lett. 546: 108-12). Akt kinase is a serine/threonine kinase activated by both phosphatidylinositol 3-kinase (PI3K)-dependent and phosphatidylinositol 3-kinase (PI3K)-independent mechanisms and negatively regulated by src-homology-2 domain-containing inositol phosphatases (SHIP-1/2) and PTEN phosphatase. Akt can either negatively or positively regulate downstream targets by altering their enzymatic activity or cellular localization. Akt is activated mainly as a consequence of activation of the second messenger phospholipid kinase, PI3K, although PI3K/PDKI-independent mechanisms of Akt activation do exist. Akt regulates its downstream targets by altering their enzymatic activity or cellular localization. The Akt substrate GSK3P is upstream of metabolic responses and is involved in the regulation of proliferative and anti-apoptotic pathways. The enzymatic activity of GSK3β isoforms is inhibited by Akt-mediated phosphorylation (Jope and Johnson, 2004, Trends Biochem. Sci. 29: 95-102). The Forkhead family of transcription factors, also known as the Foxo protein family are Akt substrates that have been well documented to play a role in programmed cell death. The Forkhead proteins are sequestered in the cytoplasm by 14-3-3 proteins when phosphorylated by Akt, preventing them from fulfilling their function as pro-apoptotic transcription factors (Franke et al., 2003, Oncogene. 22: 8983-98; Scheid et al., 2003, FEBS Lett. 546: 108-12). IGF-1 protects cells from glucocorticoid induced apoptosis by activating the PI3K pathway, and inducing the phosphorylation and inactivation of the Forkhead family member, FKHRLI. Inhibition of FKHRLI results in the loss of ability to inhibit cellular proliferation and induce apoptosis (Qiang et al., 2002, Blood. 99: 4138-46).

The present invention shows that 8-amino-adenosine is a novel therapeutic for the treatment of hematological malignancies. In particular, the inventors of the invention herein show that 8-amino-adenosine can be used for the treatment of myeloma and multiple myeloma. Of significance, 8-amino-adenosine has been found to be cytotoxic to multi-drug resistant myeloma cells.

8-amino-adenosine is also herein shown to affect key pathways such as the p38 MAP kinase, ERK1/2, and Akt pathways. The correlation of decrease in phosphorylation of key proteins in these pathways and myeloma cell cytotoxicity provides the foundation for new useful methods of identifying hematological cancer drug candidates as well as identifying patients likely to respond effectively to such drugs.

### SUMMARY OF THE INVENTION

The invention encompasses treating a patient diagnosed with a hematological malignancy such a myeloma, lymphoma or leukemia with a therapeutically effective amount of 8-amino-adenosine. 8-amino-adenosine can be used in conjunction with other therapeutics to increase the efficacy and safety of the anti-cancer treatment. A pharmaceutical composition containing 8-amino-adenosine can also be used to treat a patient suffering from a reoccurring hematological malignancy and/or multi-drug resistant malignancy.

8-amino-adenosine can also be used to ameliorate or prevent a symptom or condition associated with myeloma, lymphoma or leukemia. In one embodiment, 8-amino-adenosine is administered to a patient diagnosed with myeloma for the improvement or prevention of myeloma-related conditions such as hypercalcemia, osteoporosis, osteolytic bone lesions, bone pain, unexplained bone fractures, anemia, renal damage, amyloidosis, diffuse chronic infection, weight loss, nausea, loss of appetite and mental confusion.

The present invention also includes methods of treating a subject diagnosed with myeloma, lymphoma or leukemia by administering a nucleoside analog drug to the patient at a time and dosage sufficient to substantially reduce phosphorylation of one or more of MKK3, MKK6, p38 MAP kinase, ERK1, ERK2, Akt kinase, and downstream signaling molecules thereof. In one embodiment, the patient is suffering from a reoccurring and/or drug resistant form of cancer.

The administration of 8-amino-adenosine or a nucleoside analog drug according to the methods of the present invention can result in clinical findings associated with efficacious treatment of the cancer, including, for instance, a decrease in quantity of M protein in the serum or Bence-Jones proteins in the urine of a patient suffering from myeloma.

In another embodiment of the present invention, the efficacy of an anti-cancer nucleoside analog can be assessed for a patient suffering from a hematological cancer by isolating cells from the patient, treating the cells in vitro with the nucleoside analog drug and measuring phosphorylation of one or more proteins of MKK3, MKK6, p38 MAP kinase, ERK1/2 and Akt kinase and downstream signaling molecules thereof, wherein a measured decrease in phosphorylation is indicative that the patient will respond to treatment with the drug.

The present invention also encompasses a method for screening a drug candidate for efficacy in treatment of a hematological malignancy, such as myeloma, by treating cells with the compound in vitro and measuring phosphorylation levels of one or more proteins. For instance, cultured myeloma cells can be treated with the drug candidate and phosphorylation of the cells measured to determine if the drug is efficacious for treatment of myeloma. Cultured cells used in this embodiment can be selected for multi-drug resistance and/or steroid resistance.

The methods of the invention can also include additional steps to assess the efficacy of the drug candidate to treat hematological cancers such as steps to measure PP2A phosphatase activity, apoptosis, cell proliferation and caspase activation.

### BRIEF DESCRIPTION OF THE DRAWINGS

FIGS. 1A, 1B, 1C and 1D are blots showing protein from myeloma cells treated with 8-amino-adenosine and probed with antibodies to phosphorylated and total (phosphorylated and non-phosphorylated) key pathway proteins.

FIG. 2 is a graph showing cell cycle by flow cytometry for MM.1 S cells incubated with 8-amino-adenosine for 0.5, 1, 2, 4 and 24 hours.

FIGS. 3A and 3B are blots showing protein from MM.1 S myeloma cells incubated with various nucleoside analogs and probed with antibodies to phosphorylated p38 MAP kinase.

FIG. 4 is a blot and results of an ATP assay which show the effect of ATP depletion on p38 MAPK phosphorylation levels in MM.1 S cells.

FIGS. 5A and 5B are blots showing the effect of 8-amino-adenosine in MM.1 S cells on MKP-1 and PTEN (phosphorylated and total) levels, respectively.

FIGS. 5C and 5D are blots showing the effect of 8-amino-adenosine and okadaic acid treatment in MM.1 S cells on phosphorylated p38 MAPK and total p38 MAPK.

FIG. 6 are blots showing the effect of 8-amino-adenosine in MM.1 S cells on caspase 8 and caspase 9.

### DETAILED DESCRIPTION OF THE INVENTION

The present invention describes novel methods of treating hematological diseases such as myeloma with 8-amino-adenosine (8-NH₂-Ado). The inventors of the present invention have found that 8-amino-adenosine can be used to treat multi-drug resistant and steroid resistant myeloma cells and that the drug exerts a differential effect on normal versus malignant cells making it an ideal therapeutic for hematological malignancies.

The inventors of the present invention also made the surprising discovery that 8-amino-adenosine causes a rapid and dramatic loss of phosphorylation of several important signaling proteins including ERK1/2, p38 MAPK, and Akt kinase, whereas other known pyrimidine and purine analog drugs do not alter phosphorylation levels. Although a number of cellular proteins are affected by 8-amino-adenosine, the phosphorylation status of several other signaling molecules including JNK, PKC-8 and the STAT proteins is unaltered with 8-amino-adenosine treatment, indicating that the decrease in phosphorylation caused by 8-amino-adenosine is a not a global event, but rather, a specific effect. In addition, cells depleted of ATP independent of 8-amino-adenosine do not exhibit the same decrease in phosphorylation of vital cellular proteins. Therefore, the significant shifts in endogenous ATP pools caused by 8-amino-adenosine treatment cannot account for the changes in phosphorylation levels.

As used herein, “blood cancer”, “hematological malignancy”, “hematological cancer”, “hematopoietic malignancy” and “hematopoietic cancer” refer to a blood-related diseases, including but not limited to leukemia, lymphoma, and myeloma and specific disease types thereof such as multiple myeloma (MM), Waldenstrom's macroglobulinemia, heavy chain disease, acute myelogenous leukemia (AML), acute lymphocytic leukemia (ALL), chronic myelogenous leukemia (CML), chronic lymphocytic leukemia (CLL), hairy cell leukemia, promyelocytic leukemia, myelomonocytic leukemia, monocytic leukemia, Hodgkin's lymphoma, non-Hodgkin's lymphoma (small-cell type, large-cell type, and mixed-cell type), and Burkitt's lymphoma.

Myeloma and multiple myeloma are used interchangeably herein. As one of skill in the art would appreciate, the present invention applies equally to myeloma and the sub-type multiple myeloma. Myeloma may be present at one site in the body or at multiple sites in the body, i.e., as multiple myeloma.

As used herein, “nucleoside analog drug” refers to a nucleoside containing compound. Nucleoside analog drugs of the present invention include but are not limited to 8-amino-adenosine. “Drug” and “compound” are used interchangeably herein and refer to a nucleoside analog drug such as 8-amino-adenosine.

As used herein, 8-amino-adenosine (8-NH₂-Ado) is an adenosine analog with a ribose sugar and amine group at the 8-position of the adenine base. A skilled artisan would appreciate that similar and/or related compounds, for instance, compounds of a similar structure and function, could also be used with the methods of the present invention for the treatment of hematological diseases such as myeloma. Thus, the present invention applies to methods using 8-amino-adenosine and variants thereof.

As used herein, “therapeutically effective dose” and “therapeutically effective amount” refer to dosage that is effective for the treatment of a hematological malignancy. A therapeutically effective amount can be a dosage sufficient for the alleviation, i.e., reduction, of one or more of the symptoms or clinical features associated with a hematological malignancy including but not limited to hypercalcemia, osteoporosis, osteolytic bone lesions, bone pain, unexplained bone fractures, anemia, renal damage, amyloidosis, diffuse chronic infection, weight loss, nausea, loss of appetite, infection, bleeding, and mental confusion.

A therapeutically effective amount can also be a dosage sufficient to quantitatively and/or qualitatively modulate clinical indicators of malignancy, i.e., laboratory findings, such that a skilled artisan would infer an improvement in the patient's overall condition. As used herein, “modulate” refers to an alteration such as an increase or decrease in the measured clinical indicator. Such indicators of a quantitative nature would be preferably reduced or increased by a statistically significant amount as appreciated in the art. Clinical indicators include but are not limited to a substantial increase or decrease in number of cells, the presence of cells of abnormal morphology, the presence of abnormal chromosomes in cells (e.g. Philadelphia chromosome in CML), biochemical abnormalities, and hypercellular bone marrow.

Clinical indicators of myeloma include the presence of serum and urine M proteins, the presence of Bence-Jones proteins in the urine, plasma cells of abnormal morphology, i.e., “myeloma cells”, and an overall increase in number of plasma cells. As used herein, “M protein” is defined as known in the art and refers to monoclonal immunoglobulins of a single type in a patient. In one embodiment, a therapeutically effective amount of drug, such as 8-amino-adenosine, for the treatment of myeloma results in at least about a 10% reduction in measured M protein levels, at least about a 20% reduction in measured M protein levels, at least about a 30% reduction in measured M protein levels, at least about a 40% reduction in measured M protein levels, at least about a 50% reduction in measured M protein levels, at least about a 60% reduction in measured M protein levels, at least about a 70% reduction in measured M protein levels, at least about an 80% reduction in measured M protein levels, at least about a 90% reduction in measured M protein levels, at least about a 95% reduction in measured M protein levels, or at least about a 99% reduction in measured M protein levels. M proteins can be measured by methods known in the art including but not limited to serum electrophoresis and immunofixation. M proteins measured by serum electrophoresis can be identified by the presence of a sharp peak in the gamma-globulin region in an electrophoretogram.

In another embodiment, a therapeutically effective amount of drug, such as 8-amino-adenosine, for the treatment of myeloma results in at least about a 10% reduction in measured Bence-Jones proteins, at least about a 20% reduction in measured Bence-Jones proteins, at least about a 30% reduction in measured Bence-Jones proteins, at least about a 40% reduction in measured Bence-Jones proteins, at least about a 50% reduction in measured Bence-Jones proteins, at least about a 60% reduction in measured Bence-Jones proteins, at least about a 70% reduction in measured Bence-Jones proteins, at least about an 80% reduction in measured Bence-Jones proteins, at least about a 90% reduction in measured Bence-Jones proteins, at least about a 95% reduction in measured Bence-Jones proteins, or at least about a 99% reduction in measured Bence-Jones proteins. Bence-Jones proteins, as used herein, are 25, known in the art and refer to a light chain fragment of an immunoglobulin. Bence-Jones proteins can be measured in the serum and urine by methods known in the art.

The present invention also includes a therapeutically effective amount of drug, such as 8-amino-adenosine, for the treatment of myeloma wherein the therapeutically effective amount results in a statistically significant decrease in number of myeloma cells (abnormal plasma cells) or plasma cells in the bone marrow of a patient. The terms “myeloma cells” and “plasma cells” are used interchangeably herein when referring to a subject with myeloma. Unless stated herein that plasma cells are from a normal subject, “plasma cells” should be interpreted as referring to myeloma cells.

The morphology and number of plasma cells can be determined by methods of biopsy as known in the art. In one embodiment of the present invention, a therapeutically effective amount of drug, such as 8-amino-adenosine, results in a least about a 5% reduction in number of plasma cells, at least about a 10% reduction in number of plasma cells, at least about a 20% reduction in number of plasma cells, at least about a 30% reduction in number of plasma cells, at least about a 40% reduction in number of plasma cells, at least about a 50% reduction in number of plasma cells, at least about a 60% reduction in number of plasma cells, at least about a 70% reduction in number of plasma cells, at least about a 80% reduction in number of plasma cells, at least about a 90% reduction in number of plasma cells, at least about a 95% reduction in number of plasma cells, or at least about a 99% reduction in number of plasma cells.

As used herein, “at least about” refers to an approximate minimal amount.

As used herein, “time and dosage sufficient” refers to the timing of administration of a drug and amount of drug administered that is required to achieve a substantial reduction in one or more clinical symptoms of hematological malignancy, or a reduction in phosphorylation of one or more of the proteins MKK3, MKK6, p38 MAP kinase, ERK1, ERK2, Akt kinase, and downstream signaling molecules thereof. A time and dosage is not sufficient, for instance, if it does not result in substantial reduction in phosphorylation of one or more of the specified proteins. A skilled artisan would appreciate that the time and dosage sufficient to achieve substantial reduction of phosphorylation of the specified proteins varies based on the stage of the disease, the health of the patient, the timing of the administration of the drug, and the drug dosage.

As used herein, “substantial reduction” in phosphorylation is a reduction in phosphorylation that is sufficient to slow or stop the progression of a hematological malignancy. In one embodiment, a substantial reduction is a statistically significant quantitative reduction in phosphorylation. A substantial reduction in phosphorylation may be at least about a 1% reduction, at least about a 5% reduction, at least about a 10% reduction, at least about a 15% reduction, at least about a 20% reduction, at least about a 25% reduction, at least about a 30% reduction, at least about a 40% reduction, at least about a 50% reduction, at least about a 60% reduction, at least about a 70% reduction, at least about a 80% reduction, at least about a 90% reduction, at least about a 95% reduction, or at least about a 99% reduction in phosphorylation.

As used herein, “patient” and “subject” are used interchangeably. A patient or subject is an animal that has been diagnosed with a hematological malignancy. The animal may be a mammal and is preferably a human. An animal of the present invention includes but is not limited to human, canine, feline, bovine, primate, murine, and rat.

“MKK3”, “MKK6”, and p38 MAP kinase are members of the p38 pathway. As used herein, “downstream signaling molecules” of MKK3, MKK6 and p38 MAPK are molecules which undergo a change in phosphorylation as a result of a decrease in phosphorylation of MKK3, MKK6, and p38 MAP kinase, including but are not limited to ATF-2, p36 MAP kinase, CHOP, MEF2, Elk-1, Myc, Max, Stall, MSK-1, MAPKAPK-2, MNK1, MNK2, PRAK, and Histone H3. p38 MAP kinase and p38 are used interchangeably herein.

A daily dose of 8-amino-adenosine in an amount, ranging from 500 to 2500 mg/m², can be administered to cancer patients in need of treatment, at least once and up to five days per week for at least two weeks in a two month period. The method can be practiced in a variety of embodiments; in general, the lower the dose administered within the therapeutically effective range, the more frequently the dose is administered. In one embodiment, a daily dose of 500 mg/m² is administered at least five days per week for at least two weeks in a two month period. In another embodiment, a higher dose is employed, and the dose is administered less often. In one embodiment, a daily dose of 2500 mg/m² is administered once per week for at least two weeks in a two month period.

In one embodiment, the therapeutically effective dose of 8-amino-adenosine is administered such that the week in which the 8-amino-adenosine is administered is followed by a 14 to 28 day period in which no 8-amino-adenosine is administered, which period is followed by another week of treatment with 8-amino-adenosine. A period of one week of treatment followed by two to four weeks of no treatment with 8-amino-adenosine is termed a “cycle of treatment.” Generally, at least two cycles of treatment will be administered. In other embodiments, up to six or more cycles of treatment will be administered.

In another embodiment, the therapeutically effective dose of 8-amino-adenosine is administered at least once and up to three or more, including five, days per week for one week, at least two consecutive weeks, at least three consecutive weeks, at least four consecutive weeks, at least four consecutive weeks, at least five consecutive weeks, or at least six consecutive weeks. In this embodiment, the patient is administered the therapeutically effective dose for consecutive weeks until a dose limiting toxicity occurs.

Thus, in one aspect, methods are provided for treating cancer in a subject, comprising administering to the subject an effective amount of 8-amino-adenosine. Administration of 8-amino-adenosine as provided herein can be effected by any method that enables delivery of the 8-amino-adenosine to the site of action. In some embodiments, the 8-amino-adenosine comes into contact with the hematological cancer cells or tumor tissue via circulation in the bloodstream. To place the 8-amino-adenosine in contact with cancer tissues or cells, suitable methods of administration include oral routes, intraduodenal routes, parenteral injection (including intravenous, subcutaneous, intramuscular, intravascular or infusion), topical, and rectal routes. Depending on the type of hematological cancer being treated and the route of administration, certain routes of administration, such as administration by intravenous infusion during a period ranging from one to eight hours, are preferred.

The amount of the 8-amino-adenosine administered within the dose range described herein is dependent on the subject being treated, the type and severity of the cancer, localization of the cancer, the rate of administration, the disposition of the 8-amino-adenosine (e.g., solubility and cytotoxicity) and the discretion of the prescribing physician. In some instances, dosage levels below the lower limit of the afore range may be more than adequate, while in other cases still larger doses may be employed without causing any harmful side effect, particularly if such larger doses are first divided into several small doses for administration throughout the day.

### Disorders to be Treated

Methods and compositions generally useful in the treatment of cancer in humans and other mammals in need of such treatment are provided. These methods comprise administering a therapeutically effective amount of a nucleoside analog drug such as 8-amino-adenosine or a pharmaceutically acceptable salt thereof either alone or in combination with a therapeutically effective amount of one or more additional anti-cancer compounds. The methods and compositions can be used to treat hematological malignancies, including but not limited to myeloma, multiple myeloma, Waldenstrom's macroglobulinemia, heavy chain disease, acute myelogenous leukemia, acute lymphocytic leukemia, chronic myelogenous leukemia, chronic lymphocytic leukemia, hairy cell leukemia, promyelocytic leukemia, myelomonocytic leukemia, monocytic leukemia, Hodgkin's lymphoma, non-Hodgkin's lymphoma (small-cell type, large-cell type, and mixed-cell type), and Burkitt's lymphoma. In one embodiment of the invention, 8-amino-adenosine is used to treat myeloma and multiple myeloma.

The methods and compositions can also be used to treat hematological malignancies that have metastasized. For instance, 8-amino-adenosine can be used treat myeloma which has spread to multiple locations in the bone.

In one embodiment of the present invention, a nucleoside analog drug such as 8-amino-adenosine is used to treat a hematological malignancy that is multi-drug resistant. For instance, myeloma and non-Hodgkin's lymphoma frequently become drug resistant. Myeloma can become resistant to current treatments known including but not limited to thalidomine and proteasome inhibitors such as bortezomib (Velcade). Nucleoside analog drugs of the present invention used alone or in combination with other anti-cancer therapeutics at a therapeutically effective dose can be used to treat a patient diagnosed with a multi-drug resistant hematological malignancy.

8-amino-adenosine can be co-administered in combination with other anti-cancer and anti-neoplastic agents. When employed in combination with one of these agents, the dosages of the additional agent are either the standard dosages employed for those agents or are adjusted downward or upward from levels employed when that agent is used alone. Thus, the administration of 8-amino-adenosine can allow the physician to treat cancer with existing drugs, but at a lower concentration or dose than is currently used, thus ameliorating the toxic side effects of such drugs. Alternatively, the administration of 8-amino-adenosine may allow a physician to treat cancer with existing drugs at a higher concentration or dose than is currently used. The ability to decrease or increase the dosage of another anti-cancer therapeutic is crucial for the treatment and prevention of reoccurring hematological malignancies in which a high dosage of an anti-cancer drug may result in undesirable side effects or death. One of ordinary skill in the art would appreciate that the determination of the exact dosages for a given patient varies, dependent upon a number of factors including the drug combination employed, the particular disease being treated, and the condition and prior history of the patient.

Specific dose regimens for known and approved anti-neoplastic agents are given, for example, in the product descriptions found in the current edition of the Physician's Desk Reference, Medical Economics Company, Inc., Oradell, N.J. Illustrative dosage regimens for certain anti-cancer drugs are also provided below. Those of skill in the art will recognize that many of the known anti-cancer drugs discussed herein are routinely used in combination with other drugs. In accordance with the methods described herein, 8-amino-adenosine can be co-administered in such multiple drug treatment regimens, either in addition to the agents used or in replacement of one or more of such agents.

FDA-approved cancer drugs include but are not limited to alkylators, anthracyclines, antibiotics, aromatase inhibitors, biphosphonates, cyclo-oxygenase inhibitors, estrogen receptor modulators, folate antagonists, inorganic aresenates, microtubule inhibitors, modifiers, nitrosoureas, nucleoside analogs, osteoclast inhibitors, platinum containing compounds, proteasome inhibitors, retinoids, topoisomerase 1 inhibitors, topoisomerase 2 inhibitors, and tyrosine kinase inhibitors. Anti-cancer drug from any of these classes as well as other anti-cancer drugs for the treatment of hematological malignancies can be administered prior to or after treatment with a nucleoside analog such as 8-amino-adenosine.

Useful alkylators include but are not limited to busulfan (Myleran, Busulfex), chlorambucil (Leukeran), cyclophosphamide (Cytoxan, Neosar), melphalan, L-PAM (Alkeran), dacarbazine (DTIC-Dome), and temozolamide (Temodar). In accordance with the methods described herein, 8-amino-adenosine is co-administered with an alkylator to treat a hematological malignancy. In one embodiment, the cancer is chronic myelogenous leukemia, multiple myeloma, or anaplastic astrocytoma. As one example, the compound 2-bis[(2-Chloroethyl)amino]tetrahydro-2H-1,3,2-oxazaphosphorine, 2-oxide, also commonly known as cyclophosphamide, is an alkylator used in the treatment of Stages III and IV malignant lymphomas, multiple myeloma, and leukemia. Cyclophosphamide is generally administered intravenously and is administered for induction therapy in doses of 1500-1800 mg/m.sup.2 in divided doses over a period of three to five days. For maintenance therapy, cyclophosphamide is administered in doses of 350-550 mg/m² every 7-10 days or 110-185 mg/m² twice weekly. Nucleoside analogs such as 8-amino-adenosine may be co-administered with cyclosphosphamide administered at such doses.

Useful anthracyclines include, but are not limited to, doxorubicin (Adriamycin, Doxil, Rub ex), mitoxantrone (Novantrone), idarubicin (Idamycin), varubicin (Valstar), and epirubicin (Ellence). Nucleoside analog drugs such as 8-amino-adenosine may be co-administered with an anthracycline to treat a hematopoietic malignancy. For example, the compound (8S,10S)-10-[(3-Amino-2,3,6-trideoxy-.alpha.-L-lyxo-hexopyranosyl)oxy]-8-glycoloyl-7,8,9,10-tetrahydro-6,8,1,1-trihydroxy-1-met-hoxy-5,12-naphthacenedione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia and lymphomas of both Hodgkin and non-Hodgkin types. Doxorubicin is typically administered as a single intravenous injection in a dose in the range of 60-75 mg/m² at 21-day intervals; a dose of 20 mg/m² weekly, or a dose of 30 mg/m² on each of three successive days repeated every four weeks. Nucleoside analog drugs such as 8-amino-adenosine may be co-administered with doxorubicin administered at such doses.

Useful antibiotics include, but are not limited to, dactinomycin, actinomycin D (Cosmegen), bleomycin (Blenoxane), and daunorubicin, daunomycin (Cerubidine, DanuoXome). A nucleoside analog drug such as 8-amino-adenosine may be co-administered with an antibiotic to treat hematological cancer. In one embodiment, the cancer is acute lymphocytic leukemia and other leukemias.

Useful biphosphonate inhibitors include, but are not limited to, zoledronate (Zometa). In accordance with the methods described herein, a nucleoside analog drug such as 8-amino-adenosine is co-administered with a biphosphonate inhibitor to treat a hematological cancer. In one embodiment, the cancer is multiple myeloma, bone metastases from solid tumors, or prostate cancer.

Useful folate antagonists include, but are not limited to, methotrexate and tremetrexate. Nucleoside analog drugs such as 8-amino-adenosine may be co-administered with a folate antagonist to treat hematopoietic cancer. Antifolate drugs have been used in cancer chemotherapy for over thirty years. As one example, the compound N-[4-[[(2,4-diamino-6-pteridinyl)methyl methylamino]benzoyl]-L-glutamic acid, commonly known as methotrexate, is an antifolate drug that has been used in the treatment of advanced stages of malignant lymphoma. 5-Methyl-6-[[(3,4,5-trimethoxyphenyl)-amino]m-ethyl]-2,4-quinazolinediamine is another antifolate drug and is commonly known as trimetrexate. For lymphomas, twice weekly intramuscular injections in doses of 30 mg/m.sup.2 are administered. Nucleoside analog drugs such as 8-amino-adenosine may be co-administered with methotrexate administered at such doses.

Useful microtubule “inhibitors,” which may inhibit either microtubule assembly or disassembly, include, but are not limited to, vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. Nucleoside analogs such as 8-amino-adenosine may be co-administered with a microtubule inhibitor to treat hematological malignancies. In one embodiment, the hematological malignancy is multiple myeloma. As one example, the compound 22-oxo-vincaleukoblastine, also commonly known as vincristine, is an alkaloid obtained from the common periwinkle plant (Vinca rosea, Linn.) and is useful in the treatment of acute leukemia. It has also been shown to be useful in combination with other oncolytic agents in the treatment of Hodgkin's disease. Vincristine is administered in weekly intravenous doses of 2 mg/m.sup.2 for children and 1.4 mg/m.sup.2 for adults. Nucleoside analog drugs of the invention such as 8-amino-adenosine can be co-administered with vincristine administered at such doses.

Useful nucleoside analogs that can be used in conjunction with the nucleosides of the present invention such as 8-amino-adenosine, include but are not limited to mercaptopurine, 6-MP (Purinethol), fluorouracil, 5-FU (Adrucil), thioguanine, 6-TG (Thioguanine), cytarabine (Cytosar-U, DepoCyt), floxuridine (FUDR), fludarabine (Fludara), pentostatin (Nipent), cladribine (Leustatin, 2-CdA), gemcitabine (Gemzar), and capecitabine (Xeloda). In one embodiment, the hematological malignancy is multiple myeloma or myeloma.

In another embodiment, the hematological malignancy is lymphoma or leukemia. For example, the compound 2-amino-1,7-dihydro-6H-purine-6-th-ione, also commonly known as 6-thioguanine, is a nucleoside analog effective in the therapy of acute non-pymphocytic leukemias. 6-Thioguanine is orally administered in doses of about 2 mg/kg of body weight per day. The total daily dose may be given as a single dose. If, after four weeks of dosage at this level, there is no improvement, the dosage may be increased to 3 mg/kg/day. Nucleoside analog drugs of the invention such as 8-amino-adenosine may be co-administered with 6-TG administered at such doses for treatment of acute non-pymphocytic leukemia as well as other hematological malignancies.

Useful retinoids include, but are not limited to, tretinoin, ATRA (Vesanoid), alitretinoin (Panretin), and bexarotene (Targretin). 8-amino-adenosine may be co-administered with a retinoid to treat a hematological cancer. In one embodiment, the cancer is multiple myeloma. In another embodiment, the cancer is acute promyelocytic leukemia (APL) or T-cell lymphoma.

Useful topoisomerase 1 inhibitors include, but are not limited to, topotecan (Hycamtin) and irinotecan (Camptostar). Nucleoside analogs of the present invention such as 8-amino-adenosine may be co-administered with a topoisomerase 1 inhibitor to treat cancer. Useful topoisomerase 2 inhibitors include, but are not limited to, etoposide, VP-16 (Vepesid), teniposide, VM-26 (Vumon), and etoposide phosphate (Etopophos). 8-amino-adenosine may be co-administered with a topoisomerase 2 inhibitor to treat multiple myeloma or myeloma. In another embodiment, 8-amino-adenosine may be co-administered with topoisomerase 2 for the treatment of acute lymphoblastic leukemia (ALL).

Useful tyrosine kinase inhibitors include, but are not limited to, imatinib (Gleevec). 8-amino-adenosine may be co-administered with a tyrosine kinase inhibitor to treat hematological cancer. In one embodiment, the cancer is multiple myeloma or myeloma.

Thus, methods of treating hematological cancer are provided in which a nucleoside analog of the present invention such as 8-amino-adenosine or a pharmaceutically acceptable salt thereof and one or more additional anti-cancer agents are administered to a patient. Specific embodiments of such other anti-cancer agents suitable for co-administration with 8-amino-adenosine include, but are not limited to, 5-methyl-6-[[(3,4,5-trimethoxyphenyl)]-methyl]-2,4-quinazolinediamin-e or a pharmaceutically acceptable salt thereof, (8S,10S)-10-(3-amino-2,3,-6-trideoxy-.alpha.-L-lyxo-hexopyranosyl)oxy]-8-glycoloyl-7,8,9,10-tetrahyd-ro-6,8,11-trihydroxy-1-methoxy-5,12-naphthacenedione or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pter-idinyl)methyl]-methylamino]benzoyl]-L-glutamic acid, or a pharmaceutically acceptable salt thereof; or cis-diamminedichloroplatinum (II).

In one embodiment, the other anti-cancer agent is administered at least once during one of the weeks in which a nucleoside analog of the present invention is administered. In one embodiment, the other anti-cancer agent is selected from the group consisting of purine analogs, alkylating agents, and antibiotic agents. Purine analogs include gemcitabine, fludarabine, and cladribine, and in some embodiments, these are administered with 8-amino-adenosine to a patient who has been previously treated with an alkylator.

In another embodiment, GCSF is administered at least once during one of the weeks in which 8-amino-adenosine or a nucleoside of the present invention is administered. In one embodiment, about 360 to 480 Units of GCSF are administered daily to the patient. In another embodiment, a long-acting form of GCSF, such as Neulasta, is administered.

In another embodiment, erythropoietin is administered at least once during one of the weeks in which 8-amino-adenosine is administered. In one embodiment, about 40,000 Units of erythropoietin are administered. Suitable formulations include the Epogen and ProQuist formulations; another suitable formulation, which is long-acting, is the Aranist formulation.

### Formulations

The 8-amino-adenosine composition may, for example, be in a form suitable for oral administration as a tablet capsule, pill powder, sustained release formulations, solution, suspension, for parenteral injection as a sterile solution, suspension or emulsion, for topical administration as an ointment or cream, or for rectal administration as a suppository. The 8-amino-adenosine composition may be in unit dosage forms suitable for single administration of precise dosages and will typically include a conventional pharmaceutical carrier or excipient. Exemplary parenteral administration forms include solutions or suspensions of 8-amino-adenosine in sterile aqueous solutions, for example, aqueous propylene glycol or dextrose solutions. Such dosage forms can be suitably buffered, if desired. Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the 8-amino-adenosine therein may be combined with various sweetening or flavoring agents, coloring matters or dyes and, if desired, emulsifying agents or suspending agents, together with diluents such as water, ethanol, propylene glycol, glycerin, or combinations thereof. Topical formulations of 8-amino-adenosine can be used for treatment. Such formulations can be conveniently prepared using oil-water emulsions and liposomes and may optionally include one or more additional anti-cancer agents.

Methods of preparing various pharmaceutical compositions with a specific amount of active agent are known, or will be apparent, to those skilled in this art, and can be applied to 8-amino-adenosine and the nucleoside analog drugs of this invention in view of this disclosure. For examples of suitable formulations and processes, see Remington's Pharmaceutical Sciences, Mack Publishing Company, Easter, Pa., 15.sup.th Edition (1975).

In one embodiment, the nucleoside derivative drug of the invention is formulated as a tablet or pill. The formulation may be crystalline in nature. A pharmaceutical composition may contain at least about 0.1 mg, at least about 1 mg, at least about 10 mg, at least about 100 mg, at least about 250 mg, at least about 500 mg, at least about 750 mg, at least about 1 g, at least about 3 g, at least about 5 g, or at least about 10 g of the nucleoside derivative drug. Likewise, a pharmaceutical composition may contain at least about 0.1 mg, at least about 1 mg, at least about 10 mg, at least about 100 mg, at least about 250 mg, at least about 500 mg, at least about 750 mg, at least about 1 g, at least about 3 g, at least about 5 g, or at least about 10 g of 8-amino-adenosine.

A decided practical advantage of the nucleoside analog compounds is that the compounds can be administered in any convenient manner such as by the oral, intravenous, intramuscular, topical, or subcutaneous routes. Thus, nucleoside analog drugs such as 8-amino-adenosine can be orally administered, for instance, with an inert diluent, or it can be enclosed in hard or soft shell gelatin capsules, or it can be compressed into tablets, or it can be incorporated directly with the food of the diet. For oral therapeutic administration, nucleoside analog drugs such as 8-amino-adenosine can be used in conjunction with excipients and administered in the form of ingestible tablets, buccal tablets, troches, capsules, elixirs, suspensions, syrups, wafers, and the like. Such compositions and preparations contain a therapeutically effective amount of the active agent to treat a patient with a hematological cancer as described above.

Nucleoside derivative drugs, such as 8-amino-adenosine, in the form of tablets, troches, pills, capsules, and the like may also contain the following: a binder such as gum tragacanth, acacia, corn starch, or gelatin; excipients such as dicalcium phosphate; a disintegrating agent such as corn starch, potato starch, alginic acid, and the like; a lubricant such as magnesium stearate; a sweetening agent such as saccharin; and/or a flavoring agent such as peppermint, oil of wintergreen, or cherry flavoring. When the dosage unit form is a capsule, it may contain, in addition to the above-described ingredients, a liquid carrier. Various other ingredients can be present as coatings or to otherwise modify the physical form of the dosage unit. For instance, tablets, pills, or capsules can be coated with shellac.

A syrup or elixir can contain the active compound, a sweetening agent, methyl and propylparabens as preservatives, and a flavoring such as cherry or orange flavor. Of course, any material used in preparing any dosage unit form should be pharmaceutically pure and substantially non-toxic in the amounts employed.

In addition, the nucleoside derivative drug can be incorporated into sustained-release preparations and formulations known in the art.

Nucleoside analog drugs, such as 8-amino-adenosine, can also be administered parenterally or intraperitoneally. A solution of a nucleoside analog drug as a free acid or pharmacologically acceptable salt can be prepared in water suitably mixed with a surfactant known in the art including but not limited to hydroxypropylcellulose. Dispersions can also be prepared by methods known in the art, including but not limited to the use of glycerol, liquid polyethylene glycols and mixtures thereof and oils.

Under ordinary conditions of storage and use, the pharmaceutical preparation of a nucleoside analog drug such as 8-amino-adenosine of the invention can contain one or more preservatives to prevent the growth of microorganisms.

Pharmaceutical formulations of a nucleoside analog such as 8-amino-adenosine suitable for injectable use include sterile aqueous solutions or dispersions and sterile powders for the extemporaneous preparation of sterile injectable solutions or dispersions. In all cases, the form must be sterile and, in final form, must be fluid to the extent that easy administered using a syringe. It must be stale under the conditions of manufacture and storage and must be preserved against the contaminating action of microorganisms such as bacteria and fungi. In many cases, it will be preferable to include isotonic agents, for example sodium chloride. Prolonged absorption of the injectable compositions can be brought about by the use in the compositions of agents delaying absorption, for example, aluminum monostearate and gelatin.

Sterile injectable solutions are prepared by incorporating the nucleoside analog drug of the invention in the required amount in the appropriate solvent with, optionally, various other ingredients enumerated above, followed by filtered sterilization. Generally, dispersions are prepared by incorporating the various sterilized nucleoside analog drug into a sterile vehicle which contains the basic dispersion medium and the required other ingredients from those enumerated above. In the case of sterile powders for the preparation of sterile injectable solutions, methods of preparation include but are not limited to vacuum drying and the freeze drying. These methods yield a powder of the nucleoside analog drug plus any additional desired ingredient from previously sterile filtered solution thereof.

As used herein, a “pharmaceutically acceptable carrier” includes any and all solvents, dispersion media, coatings, antibacterial and antifungal agents, isotonic agents, absorption delaying agents, and the like. The use of such media and agents for pharmaceutical active substances is well known in the art. Except insofar as any conventional media or agent is incompatible with the active ingredient, its use in the therapeutic compositions of the invention is contemplated. Supplementary active ingredients can be incorporated into the compositions of the invention.

Pharmaceutical formulations of the nucleoside analog drug of the invention, including 8-amino-adenosine, that are suitable for topical use include oil and water emulsions and liposomal formulations, as well as lotions, creams, and ointments commonly used for topical administration of drugs. The carrier can be a solvent or dispersion medium containing, for example, water, ethanol, polyol, for example, glycerol, propylene glycol, and liquid polyethylene glycol, and the like, suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various anti-bacterial and anti-fungal agents, for example, parabens, chlorobutanol, phenol, sorbic acid, thimerosal, and the like.

It is essentially advantageous to formulate parental and other compositions in dosage unit form for ease of administration and uniformity of dosage. Dosage unit form as used herein refers to physically discrete units suited as unitary dosages for the mammalian subjects to be treated; each unit containing a predetermined quantity of nucleoside analog drug calculated to produce the desired therapeutic effect in association with the required pharmaceutical carrier. The specification for the novel dosage unit forms of the invention are dictated by and directly dependent on the patient and cancer to be treated and can vary from patient to patient and cancer to cancer, but generally, a dosage unit form contains from about 0.1 mg to about 10 g of 8-amino-adenosine. Typical unit forms can contain about 0.5 to about 1 g of 8-amino-adenosine. In one embodiment, the pharmaceutical composition of the invention comprises 8-amino-adenosine and a pharmaceutically acceptable carrier, and is a sterile solution suitable for intravenous infusion in a period of time ranging from 1 to 8 hours and in which the 8-amino-adenosine is present at a concentration ranging from 5 mg/mL to 10 mg/mL. In one embodiment, the pharmaceutically acceptable carrier is 5% Dextrose Injection, USP.

### Kits

Kits are provided with unit doses of the 8-amino-adenosine, in oral and injectable dose forms. In addition to the containers containing the unit doses (either oral or injectable), these kits can contain an informational package insert describing the use and attendant benefits of 8-amino-adenosine for the treatment of hematological malignancies, in particular plasma cell malignancies such as myeloma.

### Diagnostic and Prognostic Methods

The present invention includes methods for determining whether a patient diagnosed with a hematological cancer is likely to respond to treatment with 8-amino-adenosine or other nucleoside analog drug which targets one or more of MKK3, MKK6, p38 MAP kinase, BRK1/2 and Akt kinase and downstream molecules thereof. This method provides treating cells from the patient with 8-amino-adenosine or nucleoside analog drug of the invention and measuring the phosphorylation of one or more proteins of MKK3, MKK6, p38 MAP kinase, BRK1, ERK2, and Akt kinase and downstream signaling molecules thereof, wherein a decrease in phosphorylation of one or more of the proteins is indicative that the drug will be effective for the treatment of the cancer. Suitable downstream molecules include but are not limited to ATF-2, p36 MAP kinase, CHOP, MEF2, Elk-1, Myc, Max, Stall, MSK-1, MAPKAPK-2, MNK1, MNK2, PRAK, and Histone H3.

The reduction of phosphorylation indicative that a patient will respond positively to treatment for a hematological disease such as myeloma is evidenced by a reduction in phosphorylation of one of the above-listed proteins by at least about a 1% reduction, at least about a 5% reduction, at least about a 10% reduction, at least about a 15% reduction, at least about a 20% reduction, at least about a 25% reduction, at least about a 30% reduction, at least about a 40% reduction, at least about a 50% reduction, at least about a 60% reduction, at least about a 70% reduction, at least about a 80% reduction, at least about a 90% reduction, at least about a 95% reduction, or at least about a 99% reduction compared to untreated cells.

Bone marrow cells from the patient can be extracted by biopsy using methods known in the art. Bone marrow cells include plasma cells as well as other cell types. Optionally, the immune cell of interest is further isolated. In one embodiment, the immune cells are plasma cells (myeloma cells). Particular cell types can be further isolated from the mixture of bone marrow cells using methods known in the art. In order to determine the levels of phosphorylation of the proteins with the cells, it may be necessary to lyse the cells and/or isolate proteins from the cells as known in the art.

The level of phosphorylation of one or more of the above-described proteins can be measured using any methods known in the art. In one embodiment, the method of measuring phosphorylation is a Western blot analysis. The blot can be probed with an antibody to a phosphorylated form of MKK3, MKK6, p38 MAP kinase, ERK1, ERK2, or Akt kinase and downstream signaling molecules thereof.

In one embodiment of the invention, the method is used to determine whether a patient diagnosed with myeloma or multiple myeloma will respond effectively to the treatment or will not respond to the treatment. Plasma cells are isolated from the patient and treated with 8-amino-adenosine or other nucleoside analog drug capable of decreasing levels of one or more of phosphorylation of MKK3, MKK6, p38 MAP kinase, ERK1, ERK2, or Akt kinase and downstream signaling molecules thereof.

In another embodiment of the present invention, cells from the patient treated with 8-amino-adenosine or other nucleoside analog are compared to a control such as untreated cells from the patient. Cells from the patient treated with 8-amino-adenosine or other nucleoside analog can also be compared to control cells as known in the art.

### Compound Screening Methods

The present invention includes methods of screening test compounds for efficacy in treatment of lymphoma, leukemia, and myeloma. In one embodiment, cells are treated with a compound and phosphorylation of one or more proteins of MKK3, MKK6, p38 MAP kinase, ERK1, ERK2, and Akt kinase, and downstream signaling molecules is measured. The downstream molecules include but are not limited to ATF-2, p36 MAP kinase, CHOP, MEF2, Elk-1, Myc, Max, Stall, MSK-1, MAPKAPK-2, MNK1, MNK2, PRAK, and Histone H3. A decrease in phosphorylation of one or more of the measured proteins is indicative of an efficacious treatment of the blood cancer. The decrease in phosphorylation indicative of an effective treatment is at least about a 10% decrease in phosphorylation, at least about a 20% decrease in phosphorylation, at least about a 30% decrease in phosphorylation, at least about a 40% decrease in phosphorylation, at least a 50% decrease in phosphorylation, at least about a 60% decrease in phosphorylation, at least about a 70% decrease in phosphorylation, at least about an 80% decrease in phosphorylation, at least about a 90% decrease in phosphorylation, or at least about a 99% decrease in phosphorylation compared to cells not treated with the test compound. In one embodiment, the blood cancer is myeloma.

Cells of the present invention can be cultured immune cells as known in the art. In one embodiment, the immune cells are cultured diseased cells such a myeloma cells. The cells may be multi-drug resistant including but not limited to multi-drug resistant myeloma cells. The invention also includes cells which are steroid resistant, such as steroid resistant myeloma cells. In another embodiment, the cultured cells are normal immune cells, such as normal plasma cells.

Cells of the present invention may also be cells harvested from an animal by cell harvesting and biopsy methods known in the art. In one embodiment, the animal is a human. In another embodiment, the animal is a canine, feline, rat, murine, primate, or bovine. The cells may be diseased cells such as myeloma cells or may be normal cells. Normal cells may be taken from a healthy animal. Alternatively, normal cells may be obtained from a diseased animal in which the normal cells are adjacent to diseased cells.

In one embodiment of the present invention, diseased cells are treated with a test compound and the resulting phosphorylation values as described above are compared to those of normal healthy cells treated with the same compound. In another embodiment, the diseased cells are treated with a test compound and are compared to untreated diseased cells. One of skill in the art would appreciate that a variety of controls, including positive and negative controls, can be used to confirm the ability of a test compound to treat hematological cancer such as myeloma. For instance, the phosphorylation levels of MKK3, MKK6, p38 MAP kinase, ERK1, ERK2, or Akt kinase, and downstream signaling molecules of myeloma cells treated with a test compound can be compared to either phosphorylation levels from myeloma cells treated with a compound with known effects on the phosphorylation levels of the one or more proteins or untreated myeloma cells.

In another embodiment of the present invention, phosphatase activity of PP2A of the test cells is measured using methods known in the art. An increase in phosphatase activity is indicative that the treatment will be effective for a hematological malignancy such as myeloma. One of skill in the art would appreciate that phosphatase activity of control cells, i.e., cells not treated with the compound or cells treated with a compound with known phosphatase activity, can be used to with the claimed invention. An increase in phosphatase activity of PP2A of at least about 10%, at least about 20%, at least about 30%, at least about 40%, at least about 50%, at least about 60%, at least about 70%, at least about 80%, at least about 90%, or at least about 95% is indicative of an effective treatment.

Additionally, the method of the invention can include measuring apoptosis of said myeloma cells, wherein an increase in apoptosis is indicative of an efficacious treatment for multiple myeloma. Apoptosis can be measured by assays known in the art. The level of apoptosis indicative of an efficacious treatment of a hematological cancer such as myeloma can be at least about a 10% increase in apoptosis, at least about a 15% increase in apoptosis, at least about a 20% increase in apoptosis, at least about a 25% increase in apoptosis, at least about a 30% increase in apoptosis, at least about a 40% increase in apoptosis, at least about a 45% increase in apoptosis, at least about a 50% increase in apoptosis, at least about a 60% increase in apoptosis, at least about a 70% in apoptosis, at least about an 80% increase in apoptosis, at least about a 90% increase in apoptosis, and at least about a 95% increase in apoptosis.

In another embodiment of the present invention, test cells are further assayed for cell proliferation wherein a decrease in cell proliferation is indicative of an effective treatment of a hematological cancer such as myeloma. Cells can be assayed using cell proliferation assays as known in the art. For instance, myeloma cells treated with a test compound can be assayed for cell proliferation. A decrease in phosphorylation of one or more proteins of MKK3, MKK6, p38 MAP kinase, ERK1, BRK2, and Akt kinase, and downstream signaling molecules and a decrease in cell proliferation of cells treated with the test compound is indicative that the drug is effective as treatment for myeloma. A decrease in cell proliferation of at least about 10%, at least about 20%, at least about 30%, at least about 40%, at least about 50%, at least about 60%, at least about 70%, at least about 80%, at least about 90%, or at least about 95% is indicative of a successful treatment.

The present invention includes, optionally, detecting caspase activation of the cells treated with a test drug wherein caspase activation is indicative of an efficacious treatment of a hematological malignancy. In one embodiment, the hematological malignancy is myeloma. In another embodiment, the hematological malignancy is leukemia or lymphoma.

### EXAMPLES

### Materials and Methods

### Cell Culture

The MM.1 S and MM.1 R cell lines were previously developed (Goldman-Leikin et al., 1980, J. Lab. Clin. Invest. 113: 335-45). The original cell line (MM. 1) was established from the peripheral blood of a MM patient treated with steroid based therapy. A steroid-sensitive clone (MM.1S) was isolated and subsequently, a steroid-resistant variant (MM.1R) developed by chronic exposure to glucocorticoids. RPMI 8226 cells and the multi-drug resistant derivative MDR10V MM cells were obtained from Dr. William Dalton (H. Lee Moffitt Cancer Center, Tampa, Fla.) (Bellamy et al., 1991, Cancer Res. 51: 995-1002). Cells were grown in RPMI-1640 media (Invitrogen, Baltimore, Md.) supplemented with 10% fetal bovine serum, 2 mM glutamine, 100 units/ml penicillin, 100 μg/ml streptomycin and 2.5 μg/ml fungizone in a 37° C. incubator with 5% CO₂.

### Drugs and Chemicals

8-NH₂-Ado was purchased from R. I. Chemicals, Inc. (Orange, Calif.) and 8-amino-adenosine from Bio Log (La Jolla, Calif.). Cytarabine was obtained from Sigma (St. Louis, Mo.). Fludarabine was purchased from Berlex Laboratories (Alameda, Calif.) as a sterile, lyophilized powder that was dephosphorylated to its nucleoside, F-ara-A, for in vitro studies. Gemcitabine was obtained from Eli Lilly and Co. (Indianapolis, Ind.). The kinase inhibitors SB202190 and SB203580 were purchased from Sigma (Saint Louis, Mich.), and PD98059, U0126 and LY294002 were obtained from Calbiochem (San Diego, Calif.). Okadaic acid was purchased from Alexis Biochemicals (San Diego, Calif.).

### Example 1

### Cell Proliferation Assay

The MTS assay was performed as described previously (Krett et al., Clin Cancer Res. 3: 1781-1787). Briefly, MM cells were cultured into 96 well dishes at a concentration of 25,000 cells per well and incubated with the 8-NH₂-Ado for 72 hours. Cell proliferation was determined using the MTS Cell Titer Aqᵤeoᵤs assay (Promega, Madison, Wis.), which measured the conversion of a tetrazolium compound into formazan by a mitochondrial dehydrogenase enzyme in live cells. The quantity of formazan product as measured by the amount of 490 nm absorbance is directly proportional to the number of living cells in culture. The data were expressed as the percentage of formazan produced by the cells treated with the control medium in the same assay.

### Example 2

### Immunoblotting Analysis

5×10⁶ were cells treated with 10 μM 8-NH₂-Ado for the indicated times and harvested. Cell pellets were washed with cold phosphate-buffered saline (PBS; 8.1 g NaCl, 1.14 g Na₂HPO₄, 0.22 g KCl, and 0.25 g/L KH₂PO₄) and incubated with lysis buffer (50 mM HEPES, 150 mM NaCl, 1.5 mM MgCl₂, 1 mM EDTA pH 8.0, 100 mM NaFI, 10 mM Na Pyrophosphate, 500 μM PMSF, 0.5% Triton X-100, 10% glycerol) at 4° C. for one hour. Lysates were centrifuged at 4° C. for 1 minute at 9000×g, and the supernatants were collected and stored at −20° C. Protein concentration was determined by Bio-Rad protein assay (BioRad Laboratories, Hercules, Calif.). Protein, at a concentration of 30 μg, was mixed with sample buffer (125 mM Tris, pH 6.8, 4% SDS, 20% glycerol, 100 mM Dithiothreitol (DTT), and 0.05% bromophenol blue), and fractionated on a pre-cast 8-16% Tris-Glycine gel (Invitrogen/Novex, Carlsbad, Calif.). Proteins were then transferred to a Polyvinylidene Fluoride (PVDF) membrane (Immobilon-P, Millipore, Bedford, Mass.): Following protein transfer, membranes were blocked with 5% non-fat milk in PBS-T (PBS with 0.1% Tween), incubated with the primary antibody overnight at 4° C. and subsequently with horseradish peroxidase linked secondary antibody (Amersham, Arlington Heights, Ill.). Blots were developed using ECL Plus Chemiluminescent Western Blotting Detection reagent (Amersham, Arlington Heights, Ill.) and the signal was visualized with X-ray film (Hyperfilm, Amersham, Arlington Heights, Ill.). For reprobing purposes, blots were stripped using Restore Western Blot Stripping Buffer from Pierce Biotechnology (Rockford, Ill.). Phospho-MKK3/6 (Ser189/207), phospho-p38 (Thr180/Tyr182), phospho-ATF-2 (Thr69/71), phospho-c-Raf (Ser259), phospho-MEK1/2 (Ser217/221), total MEK1/2, phospho-ERK1/2 (Thr202/Tyr204), total ERK1/2, phospho-p90RSK (Ser380), total RSK, phospho-PDK1 (Ser241), total PDKI, phospho-PTEN (Ser380), total PTEN, phospho-Akt (Ser473), total Akt, phospho-GSK-3β (Ser9), total GSK-3β, phospho-FKHRLI (Thr32)/-FKHR (Thr24), phospho-FKHR (Ser256) primary antibodies were obtained from Cell Signaling Technology (Beverly, Mass.). Total MKK3, total MKK6, total p38, total ATF-2, total c-Raf, total FKHR, total FKHRLI, phospho-JNK and total JNK were purchased from Santa Cruz Biotechnology (Santa Cruz, Calif.). Caspase 3, caspase 9, and PARP antibodies were obtained from Pharmingen (San Diego, Calif.) and anti-MKP1 from Upstate (Lake Placid, N.Y.). Anti-caspase 8 mouse serum was a generous gift of Dr. Marcus Peter (University of Chicago).

### Example 3

### Flow Cytometry

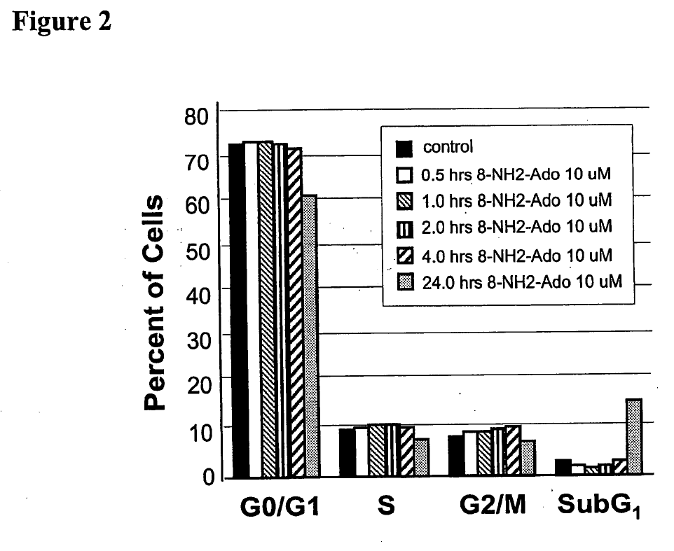

MM1 S cells were incubated with 10 μM 8-NH₂-adenosine 0.5, 1, 2, 4 or 24 hours. To determine the distribution of cells within the cell cycle, I×10⁶ MM. I S cells were pelleted (500×g for 5 minutes at 4° C.), and washed twice in ice-cold PBS, fixed in ice-cold 70% ethanol, and stored at 4° C. until analyzed. Before analysis by flow cytometry, the fixed cells were pelleted, washed in PBS, and resuspended in ice-cold flow buffer (PBS containing 0.5% Tween 20, 15 μg/mL propidium iodide, and 5 μg/mL DNase-free RNase). The stained cells were analyzed using an Epics Profile II flow cytometer (Coulter Electronics, Inc., Hialeah, Fla.). FIG. 2 provides the results of this experiment.

### Example 4

### ATP Depletion Assay

MM.IS cells were grown in dextrose-free RPMI-1640 media (Invitrogen, Baltimore, Md.) supplemented with 10% fetal bovine serum, 2 mM glutamine, 100 units/ml penicillin, 100 μg/ml streptomycin and 2.5 μg/ml fungizone. Cellular ATP levels were manipulated by the addition of either antimycin (2 μM, a mitochondrial inhibitor) or 2-deoxy-D-glucose (2-DOG, 5 mM, an inhibitor of glycolysis) from Sigma (St. Louis, Mo.) with and without varying concentrations of dextrose. Six different metabolic conditions were examined: 1) antimycin without dextrose, 2) antimycin+0.25 mM dextrose, 3) antimycin+1 mM dextrose, 4) antimycin+10 mM dextrose, 5) 2-DOG without dextrose, 6) 2-DOG+10 mM dextrose. Control cells were not subjected to ATP depletion; 10 mM dextrose was added to dextrose-free RMPI-1640. Endogenous ATP was measured in a luciferase-based assay using the ATP Determination Kit from Molecular Probes (Eugene, Oreg.) and the levels corresponding to each treatment were normalized to untreated controls (20).

### Example 5

### 8-NH₂-Ado Causes Loss of Phosphorylation of Key Signaling Molecules

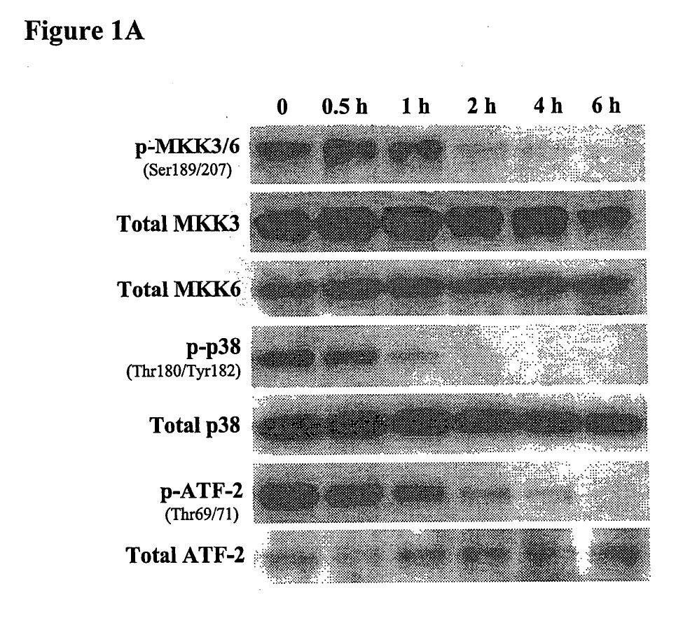

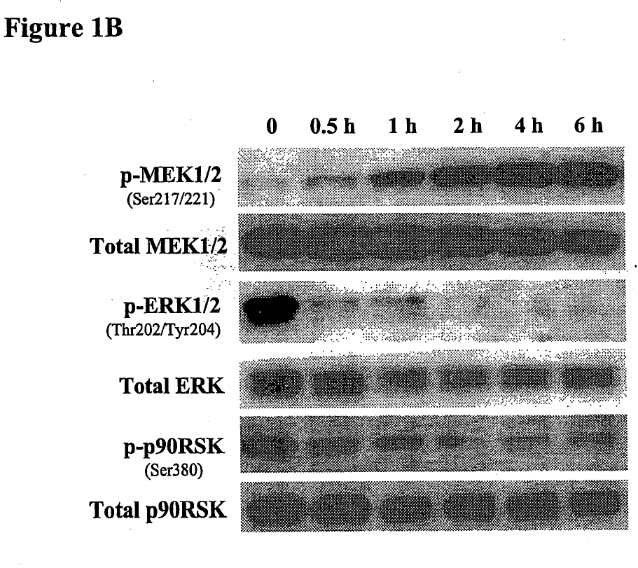

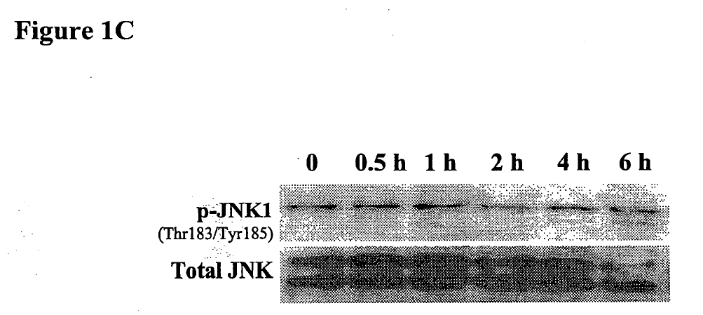

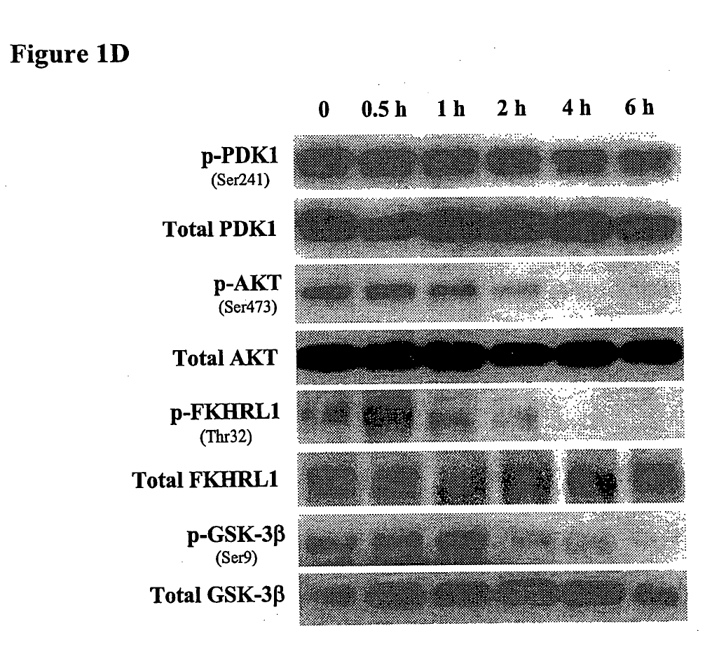

MM.1 S cells were exposed to 10 μM 8-NH₂-Ado for 0, 0.5, 1, 2, 4 and 6 hours, after which cells were lysed as previously (Example 2). 30 μg of protein was separated by gel electrophoresis, transferred to PVDF membrane, and probed with phosphorylation-specific antibodies to MKK3/6, p38 MAPK, ATF-2, MEK1/2, ERK1/2, p90RSK, JNK1, PDK1, Akt, FKHRL1 and GSK-3β. Blots were stripped and reprobed with the corresponding total protein antibodies to ensure that drug treatment does not affect total protein levels, and to ensure equal loading and transfer. FIGS. 1A, 1B, 1C and 1D provide the results of the blots.

### p38 MAPK Pathway

p38 MAPK is activated by its upstream activating kinases, MKK3 and/or MKK6. Immunoblot analysis revealed that 8-NH₂-Ado treatment induces dephosphorylation of MKK3/6 over time. Phosphorylated MKK3/6 protein levels decrease significantly by 2 hours of 8-NH₂-Ado treatment and are negligible by 6 hours of treatment. p38 phosphorylation levels are dramatically reduced by 1 hour of drug treatment, with no appreciable phosphorylation after 2 hours. The phosphorylation status of the p38 substrate, ATF-2 is also compromised, with levels of phosphorylated protein falling considerably by 2 hours of treatment (FIG. 1A). Total proteins levels for all the proteins assessed in this MAPK module remain unchanged.

### ERKI12 Pathway

Although ERKI/2 proteins undergo dramatic dephosphorylation, the phosphorylation levels of other components of the ERK pathway are not similarly affected by 8-NH₂-Ado treatment. The phosphorylation levels of the upstream ERKI/2-activating kinases MEK1/2, appear to increase, not decrease upon drug treatment, while total MEK1/2 protein levels do not change. Phosphorylation of the ERKI/2 kinases, however, decreases significantly by 30 minutes of 8-NH₂-Ado treatment and declines to negligible levels by 2 hours, while total ERKI/2 levels remain unchanged. Whereas total protein levels are unaffected, 8-NH₂-Ado treatment does seem to modestly decrease the phosphorylation level of the ERKI/2 substrate p90RSK, but this effect is not as dramatic as that observed with Erk1/2 or components of the p38 MAPK pathway (FIG. 1B).

c-Jun N-terminal Kinase (JNK)

The c jun N-terminal or stress-activated kinases (JNK/SAPK) form one subfamily of the MAPK group of serine/threonine protein kinases and are involved in cellular processes such as apoptosis. However, unlike the other MAPK proteins p38 and ERK, JNK phosphorylation is unaffected by 8-NH₂-Ado treatment (FIG. 1 Q.

### Akt Kinase Pathway

Total and phosphorylated levels of the Akt regulatory protein PDK1 remain unchanged, however, the Akt kinase dramatically loses phosphorylation upon 8-NH₂-Ado treatment Phospho-Akt levels decrease significantly by 2 hours of treatment and eventually decline further to negligible levels. The downstream targets of Akt are also similarly affected. Members of the Forkhead family of transcription factors undergo dramatic loss of phosphorylation, while total protein levels do not change. FKHRLI phosphorylation decreases dramatically by 2 hours of drug treatment, with no appreciable phosphorylation at 4 and 6 hours. FKHR phosphorylation is also similarly affected (data not shown). Phospho-OSK-3p levels diminish by 2 hours of 8-NH₂-Ado treatment and are negligible by 6 hours (FIG. 1D).

To ascertain whether the changes in phosphorylation levels of these key signaling molecules is a direct result of cell death, parallel cultures were assessed for cellular viability by cell cycle analysis. Cells undergoing apoptosis have a reduced DNA content caused by cleavage and loss of small DNA fragments. Therefore, apoptotic cells are identified as those cells in the subG₁ fraction of the cell cycle. This analysis revealed no differences between the subG, fraction of untreated cells and cells treated with 8-NH₂-Ado for up to 4 hours, indicating that the loss of phosphorylation observed by Western blotting was not due to a concomitant loss of cell viability (FIG. 2).

### Example 6

### Effect of 8-NH₂-Ado on Phosphorylation of p38 NJAPK in Various MM Cell Lines

The effect of 8-NH₂-Ado treatment on phosphorylation levels was assessed in additional myeloma cell lines, to determine whether the drug-induced alterations in protein phosphorylation occur in multiple cells lines or are limited to the MM.1 S myeloma cell line. RPMI-8226 parent myeloma cells and the multi-drug-resistant derivative MDR10V cells, and the glucocorticoid-resistant MM.1 R cells are all affected by the cytotoxic ability of 8-NH₂-Ado (12). Phosphorylation levels of p38 were assessed in these cell lines in response to 8-NH₂-Ado treatment and found to decrease in a dose-dependent manner, while total p38 levels remain unchanged. The data suggests that 8-NH₂-Ado-induced loss of protein phosphorylation is not restricted to the MM.1 S myeloma cell line (data not shown).

### Example 7

### Effect of Other Nucleoside Analogs on Phosphorylation Levels

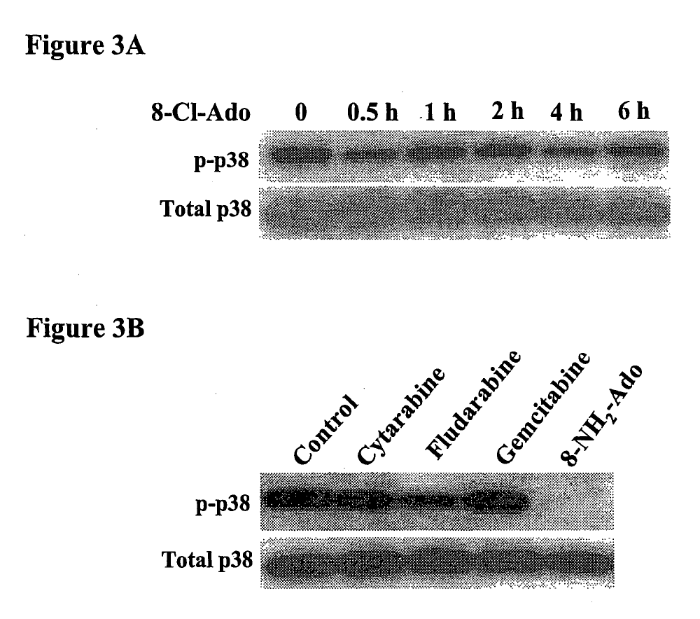

MM. I S cells were treated with 10 μM 8-chloro-adenosine for 0, 0.5, 1, 2, 4 or 6 hours or 10 μM of cytarabine, fludarabine, gemcitabine or 8-amino-adenosine for 4 hours. Cells were lysed as previously described and 30 μg of protein was separated by gel electrophoresis, transferred to PVDF membrane, and probed with phospho-p38 MAPK (Thr180/Tyr182) antibody. Blots were stripped and reprobed with total p38 MAPK antibody to ensure that drug treatment does not affect total protein levels, and to ensure equal loading and transfer. Results of representative experiments are shown in FIGS. 3A and 3B.

Not only does 8-NH₂-Ado induce a novel cellular effect by significantly altering the phosphorylation levels of key signaling molecules, but it also appears to be unique among other nucleoside analogs, both pyrimidine and purine, in its ability to do so. Although a congener of 8-NH₂-Ado, 8-chloro-Ado (8-CI-Ado), induces apoptosis in MM cells, a time course of 10 μM 8-chloro-adenosine treatment in MM. IS cells does not reveal any effect on the phosphorylation status of p38 (FIG. 3A), ERK1/2 or Akt kinase (data not shown). Fludarabine, a purine analog, and cytarabine and gemcitabine, pyrimidine analogs, have also previously been shown to be cytotoxic to MM cells. However, when used at a 10 μM concentration in MM.1 S cells for 4 hours, a time and concentration at which 8-NH₂-Ado causes a dramatic loss of phosphorylation of these kinases, they do not cause a decrease in the phosphorylation of p38 (FIG. 3B), ERK1/2 or Akt (data not shown).

### Example 8

### ATP Depletion of MMAS Cells

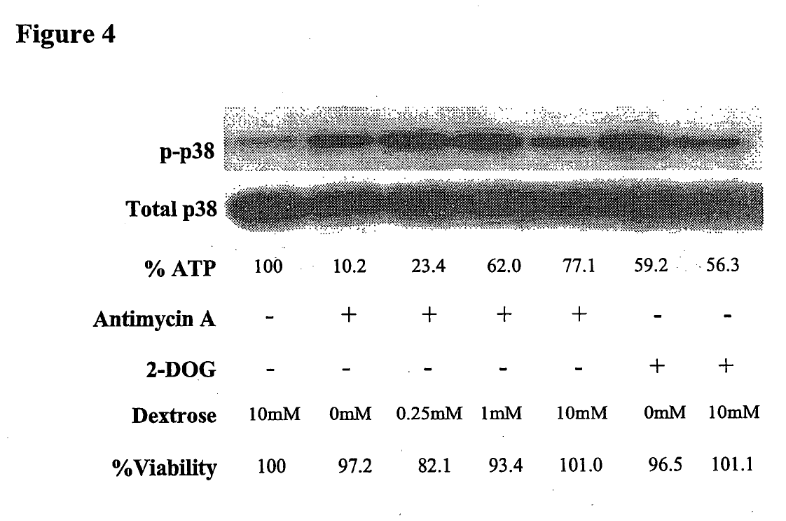

Since 8-NH₂-Ado causes dramatic shifts in endogenous ATP pools, the decrease in available ATP may have an effect on kinases or phosphatases ultimately affecting the phosphorylation of important signaling pathways in cells. To test if decreases in ATP alone are sufficient to cause the observed decreases in phosphorylation, we manipulated the cellular ATP levels by the addition of either antimycin A, which inhibits the electron transport chain, or 2-deoxyglucose (2-DOG), which inhibits glycolysis, and achieved a graded ATP-depletion by introducing increasing concentrations of dextrose. MM.1 S cells were grown in dextrose-free media and treated with 2 μM Antimycin A, 5 mM 2-DOG, and varying concentrations of dextrose for 90 minutes. Cellular ATP levels were determined using triplicate samples in a luciferase based assay and are expressed here as a percentage of untreated control. Cell viability was assessed by trypan blue exclusion and cell cycle content, and is expressed as percentage of untreated control. After treatment, cells were lysed as previously described and 30 μg of protein was separated by gel electrophoresis, transferred to PVDF membrane, and probed with a phospho-p38 MAPK (Thr180/Tyr182) antibody. Blots were stripped and reprobed with total p38 MAPK antibody to ensure that drug treatment does not affect total protein levels and to ensure equal loading and transfer. Results of a representative experiment are shown in FIG. 4; two additional studies yielded equivalent results. Additional experiments were performed using phospho-ERK1/2 and phospho-Akt (data not shown) which also did not reveal a decrease in phosphorylation. These results indicate the effect of 8-NH₂-Ado on phosphorylation of p38, ERK, Akt and other proteins in the kinase modules is not simply the result of decreased endogenous ATP levels.

### Example 9

### Effect of 8-NH₂-Ado on Cellular Phosphatases

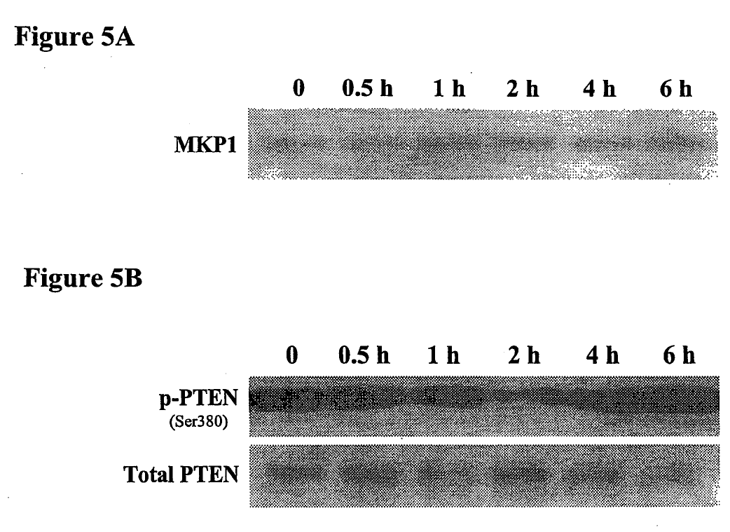

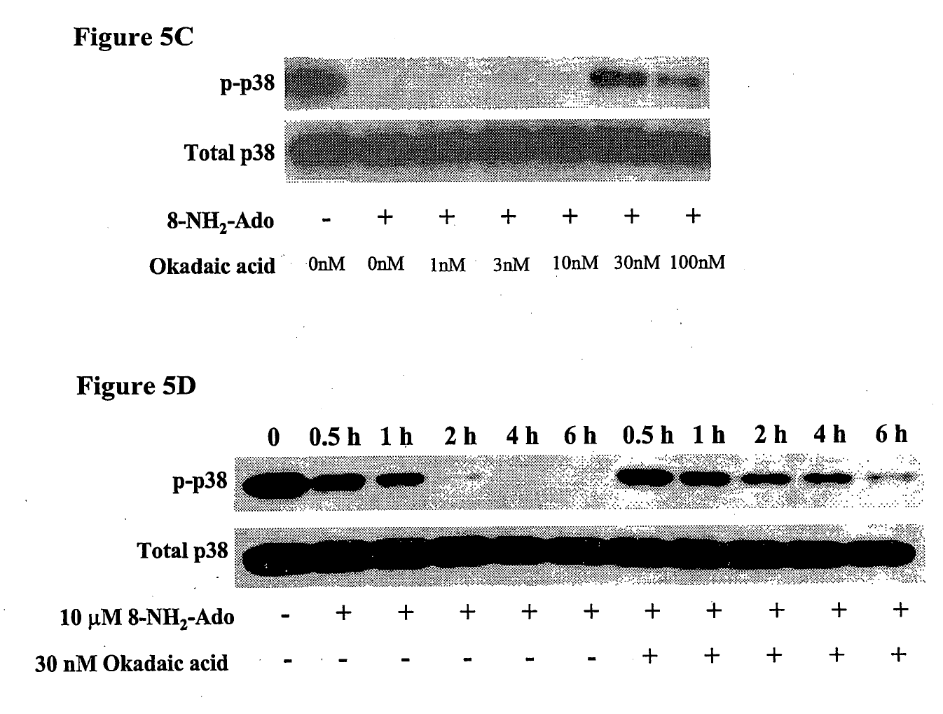

One possible mechanism for the decrease in phosphorylation of the kinase molecules and their substrates is an increase in the activity of the phosphatase(s) that regulate them. To test this hypothesis, levels of MKPI, a dual specificity phosphatase that can act to dephosphorylate p38 MAPK, were assessed MM.1 S cells were exposed to 10 μM 8-NH₂-Ado for 0, 0.5, 1, 2, 4 and 6 hours after which cells were lysed as previously described. 30 μg of protein was separated by gel electrophoresis, transferred to PVDF membrane, and probed with antibodies against MKP1. The results, as shown in FIG. 5A, suggest that this phosphatase is unlikely to be involved.

In addition, the effect of 8-NH₂-Ado treatment on PTEN, which encodes a key phosphatase involved in the negative regulation of the PI3K/Akt signaling pathway was assessed (FIG. 5B). MM.1 S cells were exposed to 10 μM 8-NH₂-Ado for 0, 0.5, 1, 2, 4 and 6 hours after which cells were lysed. 30 μg of protein was separated by gel electrophoresis, transferred to PVDF membrane, and probed with antibodies against phospho-PTEN. The blot was stripped and reprobed with the total PTEN antibody.

Like MKP1, total and phospho-PTEN levels are unaltered by 8-NH₂-Ado treatment. Although sub-cellular location plays a major role in regulation of PTEN function, phosphorylation of the C-terminal domain has also been shown to negatively regulate phosphatase activity (28, 29). Therefore, unchanged phospho-PTEN levels indicate that this phosphatase is not involved in the drug-mediated effect on protein phosphorylation.

In a parallel approach to test the involvement of cellular phosphatases, we treated MM.1 S cells with varying concentrations of the phosphatase inhibitor okadaic acid in combination with 8-NH₂-Ado for 4 hours to assess whether the serine/threonine phosphatases PP2A and PPI are involved. Cell extracts immunoblotted against phospho-p38 and total p38 antibodies showed that in the presence of 8-NH₂-Ado, there is a partial recovery of phosphorylation at a concentration of 30 nM okadaic-acid (FIG. 5C). Additionally, treatment of MM.1 S cells with okadaic acid significantly delays 8-NH₂-Ado-induced loss of p38 phosphorylation. A time course of MM.1 S cells treated with 10 μM 8-NH₂-Ado and 30 nM okadaic acid reveals that in the presence of okadaic acid, the decrease in phospho-p38 levels is delayed and still present at 6 hours, in contrast to MM.1 S cells treated with 8-NH₂-Ado alone (FIG. 5D). The 30 nM concentration of okadaic acid in cells is indicative of selective inhibition of PP2A over PPI suggesting activation of PP2A may play a role in the 8-NH₂-Ado induced decrease in phosphorylation of p38.

### Example 10

### Effect of 8-NH₂-Ado on Caspase Activation and PARP Cleavage

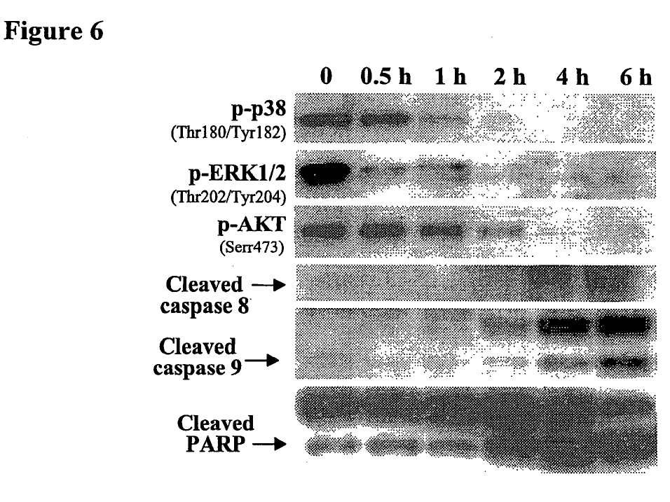

MM.1 S cells were exposed to 10 μM 8-NH₂-Ado for 0, 0.5, 1, 2, 4 or 6 hours, after which cells were lysed as previously described. 30 μg of protein was separated by gel electrophoresis, transferred to PVDF membrane, and probed with the antibodies as shown in FIG. 6. The arrows indicate the active, cleaved fragment of caspase 8 and caspase 9, and the cleaved PARP fragment. Total protein levels were also assessed to ensure equal loading and transfer (data not shown). Results of representative experiments are shown; two additional studies yielded equivalent results.

8-NH₂-Ado treatment activates the effector caspases, caspase 8 and caspase 9. FIG. 6 shows that cleaved and activated caspase 8 and caspase 9 appear between 2 to 4 hours of 10 μM 8-NH₂-Ado treatment. Cleavage of the universal caspase substrate, poly (ADP-ribose) polymerase (PARD) also occurs starting at 2 hours of drug treatment (FIG. 6). These markers of apoptosis temporally follow the loss of phosphorylation of the signaling kinases.

### Example 11

### Effect of Kinase Inhibitors on 8-NH₂-Ado-Mediated Cell Cytotoxicity

Cell proliferation assays were performed to investigate whether kinase inhibitors can modulate the effects of 8-NH₂-Ado on cellular viability. MM.1 S cells were treated with varying doses of the p38 kinase inhibitors SB202190 and S13203850, the ERK1/2 inhibitors PD98059 and U0126, and the PI3K inhibitor LY294002 alone and with 10 μM

8-NH₂-Ado. In cell viability assays, the combination of 10 μM 8-NH₂-Ado and the kinase inhibitors does not result in synergy to increase the cytotoxic effects of 8-NH₂-Ado, nor do the kinase inhibitors diminish the cytotoxic effects of 8-NH₂-Ado (data not shown).

All publications, patents, and patent applications discussed in this application are herein incorporated by reference. While in the foregoing specification this invention has been described in relation to certain embodiments thereof, and many details have been set forth for purposes of illustration, it will be apparent to those skilled in the art that the invention is susceptible to additional embodiments and that certain details described herein may be varied considerably without departing from the basic principles of the invention.

We claim:

1. A method of treating a subject diagnosed with a hematological malignancy, comprising administering a nucleoside analog drug at a time and dosage sufficient to achieve substantial reduction in phosphorylation of one or more of MKK3, MKK6, p38 MAP kinase, ERK1, ERK2, Akt kinase, and downstream signaling molecules thereof.

2. The method of claim 1, wherein said nucleoside analog drug is 8-amino-adenosine.

3. The method of claim 1, wherein said hematological malignancy is leukemia, lymphoma, or myeloma.

4. The method of claim 3, wherein said subject was in remission from said hematological malignancy and relapsed.

5. The method of claim 3, wherein said hematological malignancy is myeloma.

6. The method of claim 5, wherein the myeloma results in an increase in number of plasma cells in bone marrow of said subject.

7. The method of claim 6, wherein said plasma cells are myeloma cells.

8. The method of claim 7, wherein said myeloma cells are multi-drug resistant.

9. The method of claim 5, further comprising assaying Bence-Jones-proteins in urine of said subject, wherein a reduction or absence of Bence-Jones proteins is indicative of an effective treatment of said myeloma.

10. The method of claim 9, wherein said reduction of Bence-Jones proteins is at least about a 10% reduction in measured Bence-Jones proteins, at least about a 20% reduction in measured Bence-Jones proteins, at least about a 30% reduction in measured Bence-Jones proteins, at least about a 40% reduction in measured Bence-Jones proteins, at least about a 50% reduction in measured Bence-Jones proteins, at least about a 60% reduction in measured Bence-Jones proteins, at least about a 70% reduction in measured Bence-Jones proteins, at least about an 80% reduction in measured Bence-Jones proteins, at least about a 90% reduction in measured Bence-Jones proteins, at least about a 95% reduction in measured Bence-Jones proteins, or at least about a 99% reduction in measured Bence-Jones proteins.

11. The method of claim 5, further comprising assaying serum proteins of said subject for M protein, wherein a reduction or absence of M protein is indicative of an effective treatment of myeloma.

12. The method of claim 11, wherein said reduction of M protein is at least about a 10% reduction in measured M protein levels, at least about a 20% reduction in measured M protein levels, at least about a 30% reduction in measured M protein levels, at least about a 40% reduction in measured M protein levels, at least about a 50% reduction in measured M protein levels, at least about a 60% reduction in measured M protein levels, at least about a 70% reduction in measured M protein levels, at least about an 80% reduction in measured M protein levels, at least about a 90% reduction in measured M protein levels, at least about a 95% reduction in measured M protein levels, or at least about a 99% reduction in measured M protein levels.

13. The method of claim 11, wherein the absence of M protein is determined by immunofixation.

14. The method of claim 11, wherein said serum proteins are assayed by serum electrophoresis.

15. The method of claim 6, further comprising performing a biopsy on bone marrow of said subject to confirm a reduction in number of plasma cells indicative of an effective treatment of myeloma.

16. The method of claim 15, wherein said biopsy shows at least about a 5% reduction in number of plasma cells, at least about a 10% reduction in number of plasma cells, at least about a 20% reduction in number of plasma cells, at least about a 30% reduction in number of plasma cells, at least about a 40% reduction in number of plasma cells, at least about a 50% reduction in number of plasma cells, at least about a 60% reduction in number of plasma cells, at least about a 70% reduction in number of plasma cells, at least about a 80% reduction in number of plasma cells, at least about a 90% reduction in number of plasma cells, at least about a 95% reduction in number of plasma cells, or at least about a 99% reduction in number of plasma cells.

17. The method of claim 1, wherein said time is at least once per week for at least one week in a two month period.

18. The method of claim 17, wherein said time is at least once per week for at least two weeks in a two month period.

19. The method of claim 17, wherein said time is at least five days per week for at least one week in a two month period.

20. The method of claim 1, wherein said dosage is at least about 500 to 2500 mg/m².

21. The method of claim 1, wherein said nucleoside analog drug is administered intravenously.

22. The method of claim 1, wherein said nucleoside analog drug is administered orally.

23. The method of claim 5, wherein said administration of nucleoside analog drug ameliorates or prevents a symptom or condition associated with myeloma.

24. The method of claim 23, wherein said symptom or condition is selected from the group consisting of hypercalcemia, osteoporosis, osteolytic bone lesions, bone pain, unexplained bone fractures, anemia, renal damage, amyloidosis, diffuse chronic infection, weight loss, nausea, loss of appetite, and mental confusion.

25. The methods of claims 1-24, wherein said subject is a mammal.

26. The method of claim 25, wherein said mammal is a human.

27. A method of predicting efficacy of a nucleoside analog drug in a patient suffering from a hematological malignancy prior to treatment, comprising:
a) isolating cells from said patient;
b) treating isolated cells with the nucleoside analog drug; and
c) measuring phosphorylation of one or more proteins of MKK3, MKK6, p38 MAP kinase, ERK1, ERK2, and Akt kinase, and downstream signaling molecules thereof, wherein a decrease in phosphorylation is indicative of a favorable clinical response to said nucleoside analog drug.

28. The method of claim 27, wherein said cells are plasma cells isolated from bone marrow.

29. The method of claim 27, wherein said nucleoside analog drug is 8-amino-adenosine.

30. The method of claim 28, further comprising measuring a rate of cell proliferation of said plasma cells, wherein stabilization or reduction of said rate of cell proliferation of plasma cells is indicative that said patient will respond favorably to the nucleoside analog drug.

31. The method of claim 30, further comprising comparing said rate of cell proliferation of plasma cells to a rate of cell proliferation of normal isolated cells from bone marrow, wherein a decrease in cell proliferation of plasma cells compared to said normal cells is indicative that said patient will respond favorably to the nucleoside analog drug.

32. The method of claim 27, wherein said decrease in phosphorylation is at least about 10% less than phosphorylation of an identical protein not treated with a nucleoside analog drug, at least about 20% less than phosphorylation of an identical protein not treated with a nucleoside analog drug, at least about 30% less than phosphorylation of an identical protein not treated with a nucleoside analog drug, at least about 40% less than phosphorylation of an identical protein not treated with a nucleoside analog drug, at least about 50% less than phosphorylation of an identical protein not treated with a nucleoside analog drug, at least 60% less than phosphorylation of an identical protein not treated with a nucleoside analog drug, at least 70% less than phosphorylation of an identical protein not treated with a nucleoside analog drug, at least 80% less than phosphorylation of an identical protein not treated with a nucleoside analog drug, at least 90% less than phosphorylation of an identical protein not treated with a nucleoside analog drug, or at least 100% less than phosphorylation of an identical protein not treated with a nucleoside analog drug.

33. The method of claim 27, wherein said decrease in phosphorylation is not attributable to loss of endogenous ATP levels.

34. The method of claim 27, wherein said proteins do not undergo a change in protein levels.

35. The method of claim 27, further comprising measuring phosphatase activity of PP2A, wherein an increase in phosphatase activity of PP2A is indicative that said patient will respond favorably to said nucleoside analog drug.

36. The method of claim 27, further comprising measuring apoptosis of said plasma cells, wherein an increase in apoptosis is indicative that patient will respond favorably to said nucleoside analog drug.

37. The method of claim 26, further comprising detecting caspase activation, wherein caspase activation is indicative that said patient will respond favorably to said nucleoside analog drug.

38. A method of screening a compound for efficacy in treating multiple myeloma, comprising:
a) treating myeloma cells with said compound; and
b) measuring phosphorylation of one or more proteins of MKK3, MKK6, p38 MAP kinase, ERK1, ERK2, and Akt kinase, and downstream signaling molecules thereof
wherein a decrease in phosphorylation of said one or more proteins is indicative of an efficacious treatment for multiple myeloma.

39. The method of claim 38, further comprising measuring phosphatase activity of PP2A, wherein an increase in phosphatase activity is indicative of an efficacious treatment for multiple myeloma.

40. The method of claim 38, further comprising measuring apoptosis of said myeloma cells, wherein an increase in apoptosis is indicative of an efficacious treatment for multiple myeloma.

41. The method of claim 38, further comprising measuring cell proliferation of said myeloma cells, wherein a decrease in cell proliferation is indicative of an efficacious treatment for multiple myeloma.

42. The method of claim 38, further comprising detecting caspase activation, wherein caspase activation is indicative of an efficacious treatment of multiple myeloma.

43. The method of claim 38, wherein said myeloma cells are multi-drug resistant myeloma cells.

44. The method of claim 38, wherein said myeloma cells are steroid-resistant myeloma cells.

45. A method of treating a subject diagnosed with a hematological malignancy, comprising administering a therapeutically effective amount of 8-amino-adenosine.

46. The method of claim 45, wherein the hematological malignancy is myeloma.

47. The method of claim 46, wherein said subject was in remission from said myeloma and relapsed.

48. The method of claim 46, wherein said myeloma is multi-drug resistant.

49. The method of claim 46, further comprising assaying Bence-Jones proteins in urine of said subject, wherein a reduction or absence of Bence-Jones proteins is indicative of an effective treatment of said myeloma.

50. The method of claim 49, wherein said reduction of Bence-Jones proteins is at least about a 10% reduction in measured Bence-Jones proteins, at least about a 20% reduction in measured Bence-Jones proteins, at least about a 30% reduction in measured Bence-Jones proteins, at least about a 40% reduction in measured Bence-Jones proteins, at least about a 50% reduction in measured Bence-Jones proteins, at least about a 60% reduction in measured Bence-Jones proteins, at least about a 70% reduction in measured Bence-Jones proteins, at least about an 80% reduction in measured Bence-Jones proteins, at least about a 90% reduction in measured Bence-Jones proteins, at least about a 95% reduction in measured Bence-Jones proteins, or at least about a 99% reduction in measured Bence-Jones proteins.

51. The method of claim 46, further comprising assaying serum proteins of said subject for M protein, wherein a reduction or absence of M protein is indicative of an effective treatment of myeloma.

52. The method of claim 51, wherein said reduction of M protein is at least about a 10% reduction in measured M protein levels, at least about a 20% reduction in measured M protein levels, at least about a 30% reduction in measured M protein levels, at least about a 40% reduction in measured M protein levels, at least about a 50% reduction in measured M protein levels, at least about a 60% reduction in measured M protein levels, at least about a 70% reduction in measured M protein levels, at least about an 80% reduction in measured M protein levels, at least about a 90% reduction in measured M protein levels, at least about a 95% reduction in measured M protein levels, or at least about a 99% reduction in measured M protein levels.

53. The method of claim 51, wherein the absence of M protein is determined by immunofixation.

54. The method of claim 51, wherein said serum proteins are assayed by serum electrophoresis.

55. The method of claim 46, further comprising performing a biopsy on bone marrow of said subject to confirm a reduction in number of plasma cells indicative of an effective treatment of myeloma.

56. The method of claim 55, wherein said biopsy shows at least about a 5% reduction in number of plasma cells, at least about a 10% reduction in number of plasma cells, at least about a 20% reduction in number of plasma cells, at least about a 30% reduction in number of plasma cells, at least about a 40% reduction in number of plasma cells, at least about a 50% reduction in number of plasma cells, at least about a 60% reduction in number of plasma cells, at least about a 70% reduction in number of plasma cells, at least about a 80% reduction in number of plasma cells, at least about a 90% reduction in number of plasma cells, at least about a 95% reduction in number of plasma cells, or at least about a 99% reduction in number of plasma cells.

57. The method of claim 45, wherein said 8-amino-adenosine is administered to said subject at least once per week for at least one week in a two month period.

58. The method of claim 45, wherein said 8-amino-adenosine is administered to said subject at least once per week for at least two weeks in a two month period.

59. The method of claim 45, wherein said 8-amino-adenosine is administered to said subject at least five days per week for at least one week in a two month period.

60. The method of claim 45, wherein said 8-amino-adenosine administered to said subject dosage is at least about 500 to 2500 mg/m².

61. The method of claim 45, wherein said nucleoside analog drug is administered intravenously.

62. The method of claim 45, wherein said nucleoside analog drug is administered orally.

63. The method of claim 46, wherein said administration of 8-amino-adenosine ameliorates or prevents a symptom or condition associated with myeloma.

64. The method of claim 63, wherein said symptom or condition is selected from the group consisting of hypercalcemia, osteoporosis, osteolytic bone lesions, bone pain, unexplained bone fractures, anemia, renal damage, amyloidosis, diffuse chronic infection, weight loss, nausea, loss of appetite, and mental confusion.

65. The methods of claims 45-64, wherein said subject is a mammal.

66. The method of claim 65, wherein said mammal is a human.
